(19) United States
(12) Patent Application Publication
(10) Pub. No.: US 2021/0130417 A1
(43) Pub. Date: May 6, 2021

(54) SYSTEMS AND METHODS FOR DESIGNING SYNTHETIC ANTIMICROBIAL PEPTIDES

(71) Applicant: University of Notre Dame du Lac, Southe Bend, IN (US)

(72) Inventors: Francisco R. Fields, South Bend, IN (US); Shaun W. Lee, Mishawaka, IN (US)

(21) Appl. No.: 16/617,334

(22) PCT Filed: Jun. 1, 2018
(86) PCT No.: PCT/US2018/035718

§ 371 Date: Nov. 26, 2019

Related U.S. Application Data
(60) Provisional application No. 62/513,462, filed on Jun. 1, 2017.

(51) Int. Cl. C07K 14/195; C12Q 1/18; A01N 63/50; A01N 63/20

(52) U.S. Cl. CPC C07K 14/195; A01N 63/20; A01N 63/50; C12Q 1/18

(57) ABSTRACT

Methods for generating synthetic antimicrobial peptides include (i) identifying a peptide fragment of an antimicrobial peptide that includes a cluster of cationic residues and at least about 25% hydrophobic residues, preferably between about 40%-60% hydrophobic residues and (ii) generating a peptide variant library based on the peptide fragment by varying a hydrophobicity and charge of residues that make up the peptide fragment. Resulting synthetic peptides can include linear synthetic peptide variants of an AS-48-like bacteriocin having increased antimicrobial activity.

### CROSS REFERENCE TO RELATED APPLICATIONS

This application claims priority to and the benefit of U.S. Provisional Patent Application Ser. No. 62/513,462 filed Jun. 1, 2017 and titled “DESIGN OF MINIMAL ANTIMICROBIAL PEPTIDES FOR THERAPEUTICS.” The foregoing disclosure is incorporated herein by reference in its entirety.

### STATEMENT REGARDING FEDERALLY SPONSORED RESEARCH

The invention was made with government support under NIH 1 DP2 OD008468-01 awarded by the National Institutes of Health. The government has certain rights in the invention.

### BACKGROUND OF THE INVENTION

### Technical Field

The present disclosure relates generally to antimicrobial peptides. More specifically, the present disclosure relates to the design and generation of antimicrobial peptides, particularly those of bacterial origin.

### Description of Antimicrobial Peptides

Novel chemical scaffolds for the design of antibiotic compounds have become a priority, as bacterial species have largely become resistant to traditional antibiotics. Antimicrobial peptides (AMPs) represent an unprecedented source of chemical and functional diversity that hold enormous potential for the development of future antibiotics. AMPs have been historically studied owing to two primary roles in nature: defense against infection and niche competition. The eukaryote-derived AMPs have been extensively researched for their direct action on pathogens as components of the innate immune system. To that end, many eukaryote-derived AMPs function by accumulating on the bacterial cell surface and subsequently disrupting the bacterial membrane or cell wall, which leads to cell death.

Many of these peptides have conserved structural features, with common amphiphilic and alpha helical domains often serving as scaffolds for the design and optimization of synthetic AMPs. Strategies to improve synthetic AMP activities have generally focused on increasing the targeting affinity for anionic bacterial membranes via incorporation of basic amino acid residues and by improving their ability to penetrate lipid membrane domains via incorporation of hydrophobic residues.

Bacterial-derived AMPS, called bacteriocins, are a group of genetically encoded and ribosomally produced peptides that exist in operons containing the genes necessary for their assembly and export. Although bacteriocins are highly diverse in structure and function, nearly all bacteriocins are believed to undergo cleavage of a leader sequence from a core peptide domain as a precursor event to forming an active AMP. Bacteriocins can fundamentally be divided based on structure into class I (modified) and class II (unmodified) types.

Class I bacteriocins are subject to additional posttranslational modifications following leader sequence cleavage, including heterocyclization, glycosylation, and head-to-tail circularization. Nisin, an exemplary class I bacteriocin, is distinguished by the posttranslational installation of dehydroalanine and a thioether polycyclic lanthionine bridge. Nisin has been widely approved for use as a food preservative and is currently being researched for increased activity against infectious bacteria. Thiopeptides, a type of class I bacteriocin containing thiazole rings, have been used for the development of antibiotic lead compounds to treat Clostridium difficile infections. For example, using a naturally occurring thiopeptide as a scaffold, the compound LFF571 was designed using traditional medicinal chemistry and structure-activity relationship approaches and is now in clinical trials. Despite these significant advances, bacteriocins are still highly underrepresented as template sources for the design of AMPS.

Enterocin AS-48 is a class I circular bacteriocin produced by Enterococcus sp. and has been most commonly studied as a possible food preservative. This bacteriocin is first produced as a prepro-peptide consisting of a leader sequence and a pro-peptide. Subsequent proteolytic cleavage of the leader sequence results in a pro-peptide that undergoes head to tail macrocyclization to produce the mature, active form. Mature AS-48 consists of five alpha helices, with cationic residues clustered within helices four and five. These cationic residues have been hypothesized as being critical for the antimicrobial activity of AS-48. However, peptide variants consisting of portions of this region obtained by limited proteolysis or chemical synthesis were not found to retain full antibacterial activity.

There is no known method for quickly and efficiently identifying the regions of AS-48, or any other bacteriocin, that are central to the antimicrobial activity of the peptide. Further, it is unclear if—or how—the antimicrobial activity of bacteriocins can be improved to, for example, result in a decreased minimum inhibitory concentration for a given target bacterium or increase spectrum of antimicrobial activity (e.g., the number and/or type of bacteria sensitive to its antimicrobial effects). The complex posttranslational processing of bacteriocins is additionally problematic as it can be both time and resource intensive to synthesize bacteriocins that include non-naturally-occurring amino acids or carbohydrate modifications to various amino acids (e.g., glycosylation). Circularized bacteriocins present an additional concern, as it is unclear how modifications to the primary amino acid sequence would affect the ability or efficiency of circularization. These problems are individually, and collectively, barring progress of utilizing bacteriocins as template sources for the design of synthetic AMPs.

Accordingly, there are a number of disadvantages in the art of antimicrobial compound formulation and generation that can be addressed.

### BRIEF SUMMARY

There is a need for methods of designing and generating synthetic antimicrobial peptides that could provide a straightforward, efficient approach to optimizing known AMPs and/or identifying and optimizing novel AMPs. This need is particularly exacerbated when considering the rise and prevalence of antibiotic resistant bacterial pathogens. Current synthetic peptide design criteria and optimization protocols fail to directly address or account for complexities inherent to bacteriocin function and production, including posttranslational modifications like circularization, and fail to provide a general paradigm for identifying and/or increasing the antimicrobial activity of bacteriocins. Instead, traditional synthetic peptide design and optimization protocols require an individual peptide-by-peptide analysis driven by guesswork (e.g., functional testing of truncates and/or alanine scanning experiments) to identify functional domains with a similar approach guiding optimization.

Embodiments of the present disclosure solve one or more of the foregoing problems in the art of synthetic antimicrobial peptide design, generation, and optimization.

For example, embodiments of the present disclosure include a method for generating synthetic antimicrobial peptides can include (i) identifying a peptide fragment of an antimicrobial peptide and (ii) generating a peptide variant library based on the peptide fragment by varying a hydrophobicity and charge of residues comprising the peptide fragment. The identified peptide fragment can include a cluster of cationic residues and at least about 25% hydrophobic residues, preferably between about 40%-60% hydrophobic residues.

In one embodiment, identifying the peptide fragment includes querying a data structure having known or putative protein coding sequences, or translated amino acid sequences derived therefrom, using a query input that includes at least a portion of a known or putative bacteriocin to identify one or more homologues thereof.

In one embodiment, identifying the peptide fragment includes identifying a bacteriocin that includes the peptide fragment. The bacteriocin can have an active form such that a sequence of the peptide fragment in the active form includes only naturally occurring amino acids. Additionally, or alternatively, identifying the peptide fragment can include identifying the active form of the bacteriocin that includes the peptide fragment, wherein each residue of the peptide fragment in the active form lacks a posttranslational modification.

In one embodiment, identifying the peptide fragment includes identifying at least a portion of a class II bacteriocin or a class I circularized bacteriocin.

In one embodiment, varying the hydrophobicity and charge of residues includes iteratively substituting a lysine residue for each acidic and each polar residue within the peptide fragment to generate a primary set of peptide variants. The method can additionally include iteratively substituting a tryptophan residue for each short-chained aliphatic and each nonpolar residue within the peptide fragment to generate a secondary set of peptide variants and within each peptide variant of the primary set of peptide variants to generate a tertiary set of peptide variants.

In one embodiment, varying the hydrophobicity and charge of residues includes iteratively substituting a lysine residue for one or more aspartic acid, glutamic acid, glutamine, threonine, and/or serine residues within the peptide fragment to generate a primary set of peptide variants. The method can additionally include iteratively substituting a tryptophan residue at one or more glycine and/or alanine residues within the peptide fragment to generate a secondary set of peptide variants and within one or more peptide variants of the primary set of peptide variants to generate a tertiary set of peptide variants.

In one embodiment, the method can additionally include isolating and/or synthesizing the identified peptide fragment and/or one or more peptide variants from the peptide variant library.

In any of the foregoing embodiments, the method can additionally include assaying one or more peptide variants for increased antimicrobial activity against a target bacterium. In some instances, the assayed peptide variant demonstrates an increased antimicrobial activity against the target bacterium as compared with a baseline antimicrobial activity of the peptide fragment.

Additionally, or alternatively, varying the hydrophobicity and charge of residues can include increasing a positive charge of a peptide variant of the primary set of peptide variants and localizing the positive charge to a same face of a predicted alpha helix corresponding to a predicted secondary structure for the peptide variant. In some instances, this causes the peptide variant to become amphipathic.

Embodiments of the present disclosure can include synthetic antimicrobial peptides that are short, linear peptides made of unmodified, naturally occurring amino acids, thereby reducing the previously problematic complexity of bacteriocin circularization or posttranslational modification for antimicrobial activity. For example, a synthetic antimicrobial peptide can include any of those defined by a sequence selected from the group consisting of SEQ ID NO 6, SEQ ID NO 7, SEQ ID NO 8, SEQ ID NO 9, SEQ ID NO 10, SEQ ID NO 11, SEQ ID NO 12, SEQ ID NO 13, and SEQ ID NO 14.

In one embodiment, the synthetic antimicrobial peptide can be characterized in that it demonstrates increased antimicrobial activity against a target bacterium.

In one embodiment, the synthetic antimicrobial peptide is synthesized for use in reducing or eliminating an infection caused by the target bacterium. For example, the synthetic antimicrobial peptide can be synthesized for use in reducing or eliminating an infection of one or more components of a plant caused by Xanthamonas axonopodis.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims or may be learned by the practice of the disclosure as set forth hereinafter.

### BRIEF DESCRIPTION OF DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIG. 1 illustrates a graphical representation of a generalized paradigm for identifying and optimizing bacterial-derived antimicrobial peptides in accordance with embodiments of the present disclosure;

FIG. 2 illustrates a schematic representation of an exemplary workflow for designing synthetic antimicrobial peptides from known or novel bacteriocins.

FIG. 3 illustrates a protein sequence of the pro-peptide form of safencin AS-48 from Bacillus safensis aligned with a protein sequence of the linear synthetic peptide incorporating the C-terminal 31 amino acids of safencin AS-48, deemed syn (synthetic)-safencin.

FIG. 4A illustrates a graph of the antibacterial activity of syn-safencin on Escherichia coli, as determined by absorbance (OD600) measurements after 16 hours of growth in media with the indicated concentration of syn-safencin;

FIG. 4B illustrates a graph of the antibacterial activity of syn-safencin on Xanthamonas axonopodis, as determined by absorbance (OD600) measurements after 16 hours of growth in media with the indicated concentration of syn-safencin;

FIG. 4C illustrates a graph of the cytotoxicity of syn-safencin to human keratinocyte HaCaT cells, as determined by cell permeability assays measuring ethidium homodimer staining of cellular DNA;

FIG. 4D illustrates a graph of the hemolytic activity of various concentrations of syn-safencin on sheep red blood cells (RBCs);

FIG. 5 illustrates the amino acid sequence of syn-safencin and a predicted secondary structure of syn-safencin as represented by a model that is depicted in two separate views differing by about a 180° rotation around the longitudinal axis of the predicted alpha helix. The hydrophobic, cationic, and anionic residues of syn-safencin are shown in orange, blue, and red, respectively;

FIG. 6A is a graph illustrating the average of three circular dichroism (CD) spectroscopy scans of 25 μM syn-safencin dissolved in 9 mM sodium dodecyl sulfate (SDS) or ultrapure water;

FIG. 6B is a graph illustrating the average of three circular dichroism (CD) spectroscopy scans of 25 μM syn-safencin dissolved in 50% trifluoroethanol (TFE) or ultrapure water;

FIG. 6C is a graph illustrating the average of three circular dichroism (CD) spectroscopy scans of 5 μM syn-safencin dissolved in 5 μM lipopolysaccharide (LPS) or ultrapure water;

FIGS. 7A-7E illustrate fluorescence microscopy images (colors inverted) of propidium iodide stained E. coli treated with saline (FIG. 7A), isopropyl alcohol (FIG. 7B), 32 μM syn-safencin (FIG. 7C), 16 μM syn-safencin (FIG. 7D), or 8 syn-safencin (FIG. 7E);

FIG. 8 illustrates a graph of flow cytometry analyses of E. coli incubated in a solution containing propidium iodide following treatment with saline (brown line), isopropyl alcohol (black line), 32 μM syn-safencin (red line), 16 μM syn-safencin (blue line), 8 μM syn-safencin (purple line), 4 μM syn-safencin (green line), or 2 μM syn-safencin (yellow line);

FIG. 9 illustrates the predicted secondary structures of syn-safencin peptide variants 20, 52, 60, 90, 91, 92, 93, 94, and 96, each variant being represented by a separate model depicted in two separate views that differ by about a 180° rotation around the longitudinal axis of the predicted alpha helix. The hydrophobic, cationic, and anionic residues within each peptide variant are shown in orange, blue, and red, respectively; and

FIG. 10 illustrates the amino acid sequence alignment of the 25 residue syn-safencin peptide fragment and the syn-safencin peptide variants 20, 52, 60, 90, 91, 92, 93, 94, and 96 with residue changes between syn-safencin and each peptide variant being highlighted in red.

### DETAILED DESCRIPTION OF THE PREFERRED EMBODIMENTS

### Overview and Advantages of the Disclosed Methods and Synthetic Antimicrobial Peptides

Before describing various embodiments of the present disclosure in detail, it is to be understood that this disclosure is not limited to the parameters of the particularly exemplified systems, methods, apparatus, products, kits, and/or processes, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

The embodiments disclosed herein will now be described by reference to some more detailed embodiments, with occasional reference to any applicable accompanying drawings. These embodiments may, however, be embodied in different forms and to should not be construed as limited to the embodiments set forth herein. Rather, these embodiments are provided so that this disclosure will be thorough and complete, and will fully convey the scope of the embodiments to those skilled in the art.

As provided above, there is no known method for quickly and efficiently identifying the regions of AS-48, or any other bacteriocin, that are central to the antimicrobial activity of the peptide, and it is unclear if—or how—the antimicrobial activity of bacteriocins can be improved. Accordingly, there is a need for methods of designing and generating synthetic antimicrobial peptides that could provide a straightforward, efficient approach to optimizing known AMPs and/or identifying and optimizing novel AMPs. This need is particularly exacerbated when considering the rise and prevalence of antibiotic resistant bacterial pathogens and the failure of current synthetic peptide design criteria and optimization protocols to directly address or account for complexities inherent to bacteriocin function and production and to provide a general paradigm for identifying and/or increasing the antimicrobial activity of bacteriocins.

Embodiments of the present disclosure enable the identification and verification of linear portions of AS-48 bacteriocins as scaffolds for the design of synthetic AMPs and their subsequent optimization for charge and hydrophobicity using a peptide library approach. This results in, among other things, a general paradigm for identifying and optimizing linear peptide fragments having antimicrobial activity—and does so in a methodical, streamlined manner that is faster and less resource intensive. The methods disclosed herein additionally enable the design and synthesis of synthetic peptide variants having increased antimicrobial activity to a target bacterium and/or an increased spectrum of antimicrobial activity. Further, the disclosed methods can be applied to AS-48 homologues, specifically, to type I circularized bacteriocins and class II bacteriocins, generally, and in some respects to the design and generation of synthetic AMPs, broadly. The benefits of the disclosed methods and synthetic peptides are particularly apparent, for example, when compared with previous methods that required individual peptide-by-peptide analyses and bespoke optimization protocols that relied heavily on guesswork or unguided, brute force approaches.

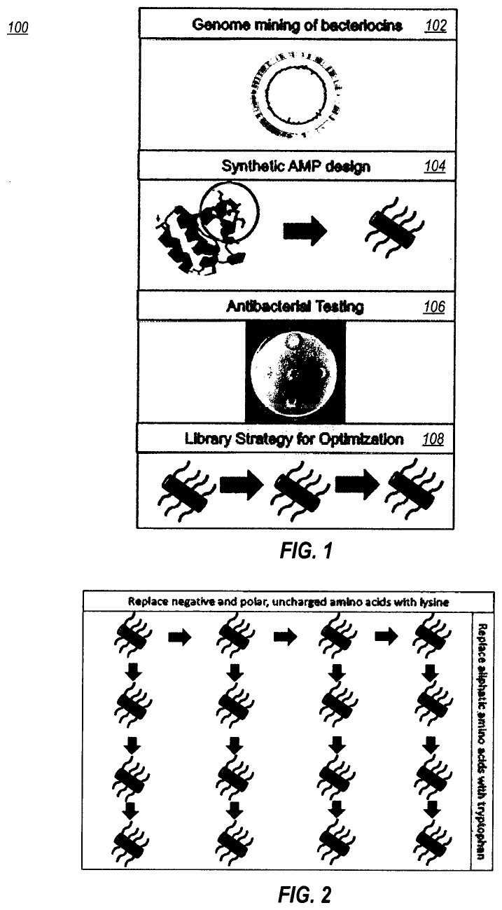

FIG. 1 illustrates a generalized paradigm 100 for identifying and optimizing bacterial-derived AMPs according to one or more embodiments of the present disclosure. The paradigm 100 includes broad steps of genome mining of bacteriocins 102, synthetic AMP design 104, antibacterial testing 106, and library strategy for optimization 108. The paradigm 100 sets forth a structured approach that, when followed, allows for the generation of a library of synthetic AMPs from scratch.

In the first broad step 102, a bacteriocin is identified. The bacteriocin can be a known and/or previously characterized bacteriocin, or alternatively, the bacteriocin can be identified by practicing step 102. If the bacteriocin is known, the amino acid sequence can be obtained at step 102 by artificially translating the coding sequence associated therewith. The coding sequence can be obtained as known in the art, including, for example, by accessing online sequence databases that include the nucleic acid sequence of the associated gene and/or the amino acid sequence of the prepro-peptide, pro-peptide, and/or mature peptide. Any of the foregoing sequences may also be obtained from disclosures provided in peer-reviewed publications.

Alternatively, if the bacteriocin is unknown, a novel bacteriocin can be identified at step 102. In one embodiment, the nucleic acid or amino acid sequence of a known or putative bacteriocin can be used to identify homologous sequences, as known in the art. For example, a nucleic/amino acid sequence of a known bacteriocin (or a relevant portion thereof) can be used in a sequence-identity-based search (e.g., BLAST) to identify homologous sequences that may putatively be bacteriocins or functional analogs thereof. As an additional example, a putative bacteriocin can be identified from an uncharacterized bacterial genome using a series of bioinformatic steps. For instance, once the nucleic acid sequence from the bacterial genome is identified, the protein coding sequences can be located and filtered away from the regulatory and other non-protein coding nucleic acid sequence. This subset of nucleic acid sequence that includes the protein coding sequences can be further refined by removing any known genes or genes having a sequence identity to known genes in a related bacterial strain, but which are not putative bacteriocin genes. The remaining subset of unknown genes can be aligned with a plurality of known bacteriocin nucleic acid sequences to identify the most likely candidate-bacteriocin genes. Additionally, or alternatively, the remaining subset of unknown genes can be artificially translated to obtain the primary structure of the associated protein. The primary structure can be used to identify protein domains similar to those found in other bacteriocins or to identify putative secondary structures of the corresponding proteins. In either case, the primary/secondary structure of the proteins can be compared with primary/secondary structures of known bacteriocins (e.g., a leader sequence, a five alpha helix domain having a cluster of cationic residues in the fourth and fifth alpha helix domains). Those proteins that best match known bacteriocins (e.g., highest sequence identity, homologous domain structure, or as otherwise known in the art) can be identified as putative bacteriocins at step 102.

Whether as part of step 102 of genome mining to identify a bacteriocin or separately performed after identifying a bacteriocin at step 102, the generalized paradigm 100 includes a step 104 of synthetic AMP design. As shown herein, AS-48—and very likely circularized class I bacteriocins generally—include a defined structural sequence within the mature AMP that is central to antimicrobial activity. Step 104 of the paradigm 100 aims to identify a peptide fragment of the larger protein sequence and to use it (or a portion thereof) as a parent sequence that is suitable for the downstream library construction of peptide variants.

In one embodiment, step 104 includes utilizing biophysical predictors and/or the overall hydrophobicity and charge of peptide fragments to identify a suitable synthetic, parent AMP. As provided above, the mode of action of many bacteriocins is to integrate a portion of the mature AMP into the bacterial membrane/cell wall, thereby disrupting it, to cause growth arrest (e.g., stasis) or cell-death. Accordingly, a suitable synthetic, parent AMP should include some consistent biophysical predictors, such as the presence of a predicted alpha helical domain, and/or an overall hydrophobicity and charge conducive to association with the amphipathic phospholipid bilayer of bacterial membranes or hydrophobic portions of Gram-negative cell wall components like LPS (e.g., the lipid A tail of LPS).

For example, a suitable peptide fragment is at least about 15 amino acids in length, preferably at least about 20-25 amino acids in length but no more than about 100 amino acids in length, preferably no more than about 30-50 amino acids in length. In some embodiments, the suitable peptide fragment is between about 15-50 amino acids, about 18-40 amino acids, about 20-35 amino acids, or preferably between about 25-30 amino acids in length.

When determining an optimal hydrophobicity of the suitable peptide fragment, at least 25% of the residues are hydrophobic. Preferably, the hydrophobicity of a suitable peptide fragment is between about 40%-60%. In some embodiments, the hydrophobicity of the suitable peptide fragment is about 50%. In some embodiments, the total number of hydrophobic residues are used to calculate the percent hydrophobicity. It should be appreciated, however, that the presence of tryptophan, phenylalanine, valine, isoleucine, and/or leucine are beneficial, as these residues typically contribute to the antimicrobial activity of the peptide. Accordingly, in some embodiments, the percent hydrophobicity can be calculated based on the number of tryptophan, phenylalanine, valine, isoleucine, and leucine residues within the peptide fragment. In some embodiments, the percentage of tryptophan, phenylalanine, valine, isoleucine, and leucine residues is at least about 20% of the total residues in the peptide fragment, preferably between about 20%-40% of the total residues in the peptide fragment.

Additionally, or alternatively, a suitable peptide fragment includes a cluster of cationic residues, as that term is defined herein. In some instances, the suitable peptide fragment includes a charge greater than or equal to +1—at pH=7—or a charge +2, +3, +4, or +5 at pH=7. In some embodiments, the charge is substantially localized to a single side or face of the peptide, preferably to a single side or face of a predicted alpha helix of the peptide fragment.

As part of step 104, identifying the suitable synthetic parent AMP can be done, for example, by scanning predicted secondary structural folds or functional domains for fragments that include the desired properties. Alternatively, a sliding window of a defined amino acid length can be used to scan protein sequences for peptide fragments having the desired properties.

Upon identifying a suitable parent AMP, the paradigm 100 can include a step 106 of antibacterial testing. The suitable parent AMP can be engineered in a plasmid and produced in a non-susceptible bacterial strain, a yeast strain, or cell culture as known in the art. Alternatively, the parent AMP can be synthesized de novo using any peptide synthesis technique, as known in the art. A series of antibacterial tests can be performed to verify the antimicrobial activity of the synthetic parent AMP, including, for example, the MIC of the synthetic parent AMP for one or more target bacteria. Antibacterial testing can be performed in solid or liquid media, as known in the art. For example, disc diffusion assays can be performed using solid media and growth inhibition assays can be performed in liquid media by measuring turbidity or live/dead staining and quantification (e.g., via microscopy or flow cytometry) following incubation with a concentration of the synthetic parent AMP.

If an effective MIC or bacterial target is unknown, a dilution series (e.g., a 2-fold, 10-fold, or combination of 2-fold and 10-fold dilutions) of the synthetic parent AMP can be reiterated in each row of a multi-well plate with controls (e.g., carrier as a negative control and chloramphenicol as a positive control) and various bacterial target strains (e.g., Gram-positive bacteria, such as Staphylococcus sp. , Streptococcus sp. , and Bacillus sp. , and Gram-negative bacteria, such as Escherichia sp. , Salmonella sp. , Xanthomonas sp. , and Pseudomonas sp. ) in each column. In such a manner, or similar method as known in the art, an effective MIC for one or more bacterial targets can be determined for the synthetic parent AMP.

On the other hand, if an effective MIC is known or suspected, and one or more bacterial targets are known or suspected, antibacterial testing to verify said antimicrobial properties of the parent AMP, as performed in step 106, may be unnecessary. In such instances, step 106 can be omitted or abbreviated (e.g., a single data point is observed instead of performing the antimicrobial testing in triplicate or series for statistical significance). In some embodiments, a narrower range of the synthetic parent AMP can be interrogated for each bacterial target, and if desired, a plurality of synthetic parent AMPS can be evaluated using the same or similar resources as in the foregoing example, thereby providing an economy of scale.

In some embodiments, however, the putative synthetic parent AMP may not have detectable antimicrobial activity. In such instances, the putative synthetic parent AMP can proceed through the paradigm 100 to determine whether antimicrobial activity can be engineered using it as a scaffold for constructing a peptide variant library. Alternatively, a different synthetic parent AMP can be identified at step 104 or at step 102, which can then proceed through steps 104 (if step 102 was performed first) and 106, as outlined above.

When a synthetic parent AMP has been identified, a strategy for, and the creation of, a synthetic peptide variant library is developed as part of the generalized paradigm 100. This step 108 of developing and optimizing a synthetic peptide variant involves a series of amino acid substitutions within the parent peptide to form one or more sets of peptide variants. At this stage, previous approaches fail to offer any predictable or consistently applicable guidance as to how a parent peptide can be altered in such a way that will, with a high likelihood of success, generate one or more optimized peptide variants. For instance, previous approaches attempted bacteriocin optimization using the entire protein instead of a portion thereof, as done here. Amino acid substitutions were, therefore, made with respect to a much larger sequence, significantly increasing the total number of possible mutations. Alternatively, some previous approaches instituted protein truncation schemes to initially identify a portion of the protein necessary for antimicrobial activity followed by ad-hoc amino acid substitutions based mostly on post-rationalized “gut feelings” or bespoke analyses of individual proteins to guide which residues were to be substituted for which other residue. These methods lack a uniform model of amino acid substitutions and are inefficient means for identifying and optimizing peptide variants having increased antimicrobial activity.

As provided herein, step 108 of the paradigm 100 provides a uniform strategy for developing a peptide variant library based on the peptide fragment (e.g., the synthetic parent AMP) by varying a hydrophobicity and charge of residues within the peptide fragment. In one embodiment, and as exemplified in FIG. 2, varying the hydrophobicity and charge of residues can include the iterative substitution of a lysine residue for each acidic residue and for each polar residue within the peptide fragment, thereby generating a primary set of peptide variants. This primary set of peptide variants can then be tested for increased antimicrobial activity, as in step 106 described above.

In one embodiment, the original peptide fragment and the primary set of peptide variants can be used as templates for the creation of secondary and tertiary sets of peptide variants, respectively. To do so, a tryptophan reside is iteratively substituted for each short-chained aliphatic residue and for each nonpolar residue within the peptide fragment or each peptide variant of the primary set of peptide variants. As above, the secondary and/or tertiary sets of peptide variants can then be tested for increased antimicrobial activity as done, for example, in step 106 described earlier.

It should be appreciated that alternative sets of peptide variants can be constructed by slightly altering one or more aspects of the foregoing optimization strategy. For example, an alternative primary set of peptide variants can be generated by iteratively substituting a lysine residue for each acidic residue but not for each polar residue within the peptide fragment. This alternative primary set of peptide variants can then be used to generate an alternative tertiary set of peptide residues by iteratively substituting a tryptophan residue for each short-chained aliphatic residue and for each nonpolar residue in each peptide variant of the alternative primary set of peptide variants. In a similar manner, the alternative tertiary set of peptide variants can include iterative substitutions of a tryptophan residue for each short-chained aliphatic residue but not for each nonpolar residue, or vice versa.

As an additional example, an alternative primary set of peptide variants can be generated by iteratively substituting a lysine residue for each polar residue but not for each acidic residue within the peptide fragment. This alternative primary set of peptide variants can then be used to generate an alternative tertiary set of peptide residues by iteratively substituting a tryptophan residue for each short-chained aliphatic residue and for each nonpolar residue in each peptide variant of the alternative primary set of peptide variants. In a similar manner, the alternative tertiary set of peptide variants can include iterative substitutions of a tryptophan residue for each short-chained aliphatic residue but not for each nonpolar residue, or vice versa.

In some embodiments, the secondary and tertiary sets of peptide variants are generated by replacing short-chained aliphatic amino acids with tryptophan. In a particular implementation of the foregoing, the secondary and tertiary sets of peptide variants are generated by iteratively substituting each glycine with tryptophan. This can significantly reduce the total number of peptide variants in the secondary and tertiary sets while concomitantly optimizing the effects of such a substitution, as a glycine residue is “floppy” and can prematurely interrupt or prevent formation of an alpha helix whereas a tryptophan residue is likely to extend or reinforce such secondary structure.

Accordingly, methods for generating synthetic antimicrobial peptides, as described herein, can include a step of generating a peptide variant library based on the peptide fragment by varying a hydrophobicity and charge of residues comprising the peptide fragment, and varying the hydrophobicity and charge of residues can be performed by any combination of iteratively replacing acidic and/or polar residues with a lysine residue and additionally, or alternatively, iteratively replacing short-chained aliphatic and/or nonpolar residues with a tryptophan residue.

The library optimization strategy (step 108) of the disclosed generalized paradigm 100 for identifying and optimizing bacterial-derived AMPS provides a much-needed systematic approach for the optimization and refinement of bacteriocin design for possible therapeutic applications. The paradigm 100 is particularly useful as a general strategy through which linear variants of many circular bacteriocins can be modified for increased activity at a reduced cost (in both time and resources). The paradigm 100 is additionally useful as a general strategy through which class II bacteriocins, and to some extent AMPs more broadly, can be identified and modified for increased activity.

### Exemplary Identification and Optimization of Linear AMP Syn-Safencin

The paradigm 100 discussed above is applicable to a wide range of situations, from further characterizing or optimizing a known bacteriocin to identifying a new bacteriocin and optimizing the antimicrobial activity of a peptide derived therefrom. As an exemplary implementation of the paradigm 100 discussed above, an AS-48 analog was identified in a newly isolated strain of Bacillus safensis from Vigna radiata seeds using a bioinformatic approach directed to mining putative bacteriocins and further optimized for increased antimicrobial activity.

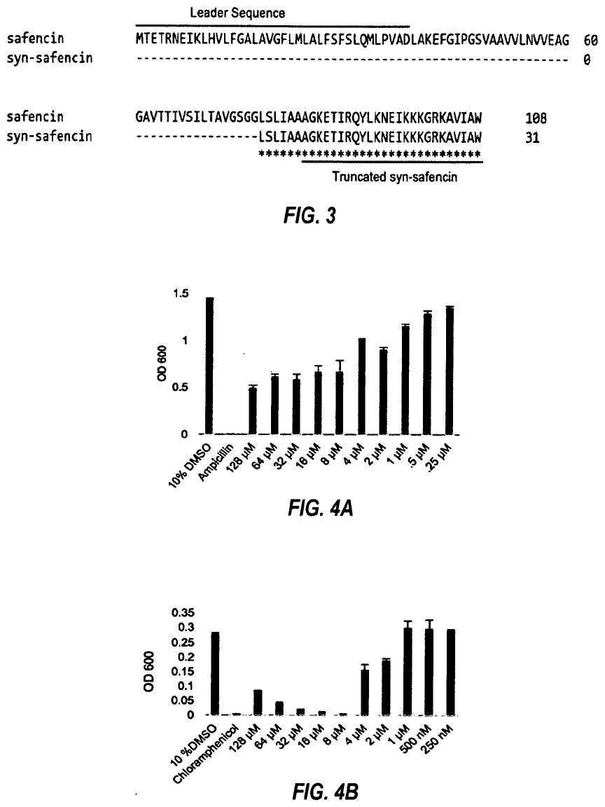

As shown in FIG. 3, the identified AS-48 analog, termed safencin AS-48 (SEQ ID NO 2), is a 108 amino acid protein having a leader sequence (SEQ ID NO 3) at the N-terminal end. An amino acid comparison of safencin AS-48 (SEQ ID NO 2) to enterocin AS-48 (SEQ ID NO 1) showed high levels of conservation. The next step (step 104) of the paradigm 100 includes identifying a peptide fragment of the larger safencin AS-48 protein sequence as a parent sequence that is suitable for the downstream library construction of peptide variants.

A linear synthetic peptide incorporating residues 39-70 of the mature AS-48 peptide from B. safensis (SEQ ID NO 4), also depicted in FIG. 3, was isolated as a putative peptide fragment for further design and analysis and designated as syn (synthetic)-safencin. Syn-safencin is marked by an overall hydrophobicity and charge that identifies it as a suitable synthetic, parent AMP. For example, syn-safencin includes the presence of a cluster of cationic residues in regions corresponding to helices four and five of the peptide and additionally has an overall hydrophobicity of 48% (e.g., between about 40%-60%) with 26% of the residues being tryptophan, phenylalanine, valine, isoleucine, or leucine.

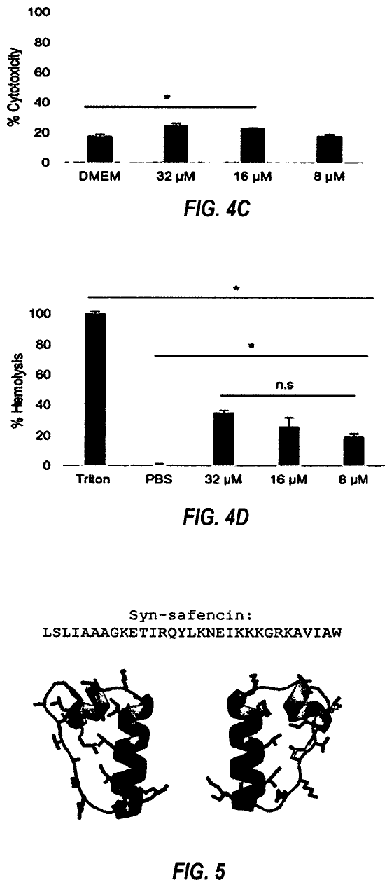

Moving to step 106 of the paradigm 100, the 31-amino acid syn-safencin was synthesized and produced to 95% purity (Genscript) and the antimicrobial activity of syn-safencin on a lab strain of E. coli as well as a known Gram-negative pathogen of Vigna radiata, X. axonopodis was assessed. As shown in FIG. 4A, syn-safencin exhibited dose-dependent bacteriostatic activity against E. coli at 16 h post incubation, and as shown in FIG. 4B, syn-safencin also demonstrated strong bacteriostatic activity against the plant pathogen X. axonopodis, with an MIC of 8 μM.

Syn-safencin was additionally evaluated for general eukaryotic cytotoxicity and hemolysis, as shown in FIGS. 4C and 4D, respectively. HaCaT cells treated for 16 h showed a slight dose-dependent increase in cytotoxicity. However, even at the highest dose of peptide (32 μM) the percent of cytotoxicity observed (˜25%) was similar to vehicle control (17%). Further, there was no significant increase in cytotoxicity observed at 8 μM of peptide, the MIC for X. axonopodis. As shown in FIG. 4B, syn-safencin displayed mild hemolytic activity at the concentrations tested, suggesting its activity as a membrane lysin.

Additional biophysical properties of syn-safencin were analyzed to further bolster the analysis of syn-safencin as a suitable peptide fragment for further optimization. Using secondary structure prediction software, syn-safencin was predicted to be an alpha helical peptide consisting of a hydrophobic and a cationic face, suggesting that the artificial peptide preserves common amphipathic, helical, and cationic features of many linear AMPs (as shown in FIG. 5).

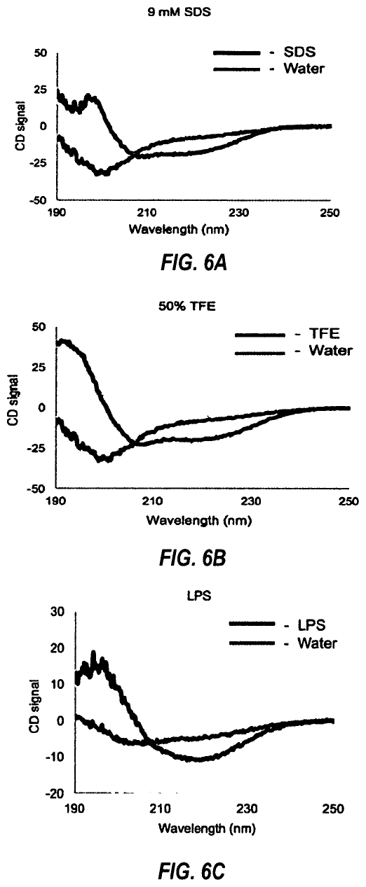

To support the modeling data, circular dichroism was used to assess the secondary structure of syn-safencin. Many cationic AMPs have been observed to adopt an alpha helix in membrane mimicking environments such as SDS and TFE, while having a random coil conformation in aqueous environments. CD analysis of syn-safencin showed a shift from a random coil conformation in an aqueous environment (ddH2O) to an alpha helical signal in both 9 mM SDS and 50% TFE (FIGS. 6A and 6B, respectively). To determine whether the peptide could adopt an alpha helical signature in the presence of bacterial membrane components, CD analysis of syn-safencin was performed in the presence of purified LPS micelles, the results of which are shown in FIG. 6C. Similar to earlier spectra of the peptide in the presence of defined membrane mimics, an alpha helical signal was observed in the presence of LPS micelles, suggesting that bacterial LPS is likely a target for the peptide.

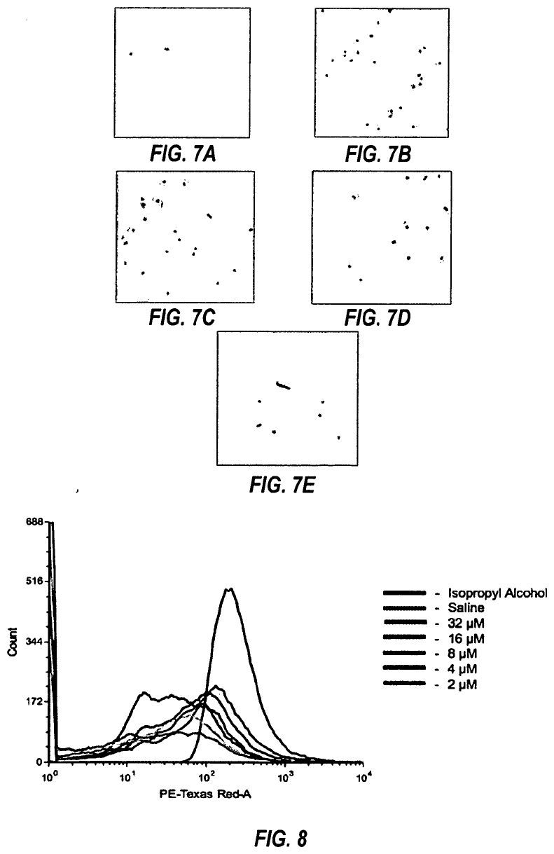

To assess whether syn-safencin could directly permeabilize bacterial membranes, PI staining was used to evaluate overall membrane permeability in the presence of peptide treatment. As observed by fluorescence microscopy shown in FIGS. 7A-7E (colors inverted), peptide treated cells showed positive staining for PI (FIGS. 7C-7E) similar to the isopropyl alcohol control (FIG. 7B) and unlike the negative saline control (FIG. 7A). In addition, E. coli cells appeared to become elongated in the presence of the peptide. An exemplary instance of this is illustrated in FIG. 7E. This elongation phenotype has been observed in E. coli as a response to membrane stress caused by cationic AMPs.

Referring to FIG. 8, illustrated is a graph of flow cytometry analyses of E. coli incubated in a solution containing propidium iodide following treatment with saline (brown line), isopropyl alcohol (black line), 32 μM syn-safencin (red line), 16 μM syn-safencin (blue line), 8 μM syn-safencin (purple line), 4 μM syn-safencin (green line), or 2 μM syn-safencin (yellow line). Flow cytometry of peptide treated cells confirmed an increase in PI positive cells in all peptide treated groups. Taken together, these foregoing results point to syn-safencin as a suitable peptide fragment for further optimization and additionally reinforce the paradigm 100 as beneficial for designing a synthetic peptide comprising a specific minimal domain within the full-length bacteriocin that retains antimicrobial activity. Furthermore, these results predict that syn-safencin is a cationic, amphipathic, helical, AMP that exerts antibacterial activity by permeabilizing the outer membrane via interaction with LPS—making it a good candidate for the generation of a synthetic variant library (step 108).

As part of step 108, a peptide variant library was designed based on the peptide fragment syn-safencin by varying the hydrophobicity and charge of syn-safencin. For ease of synthesis and library construction, an additional six amino acids were removed, to create a 25-residue truncated syn-safencin template (SEQ ID NO 5) from which variants were designed. The truncated syn-safencin (SEQ ID NO 5) maintains a cluster of cationic residues and hydrophobicity (40%) and other biophysical and antimicrobial properties of syn-safencin. A primary set of peptide variants comprising six peptide variants was created by substituting lysine for acidic and polar amino acids (i.e., truncated syn-safencin peptide variants E4K, T5K, Q8K, Y9K, N12K, and E13K).

Next, the parent peptide fragment (truncated syn-safencin) and the six variants served as templates for optimization of overall hydrophobicity, creating secondary and tertiary sets of peptide fragments, respectively. Tryptophan was substituted for nonpolar amino acids and short-chained amino acids. In total, 96 syn-safencin variants were created. These peptide variants were screened at 8 μM against coli, S. pyogenes, S. aureus, P. syringae, and P. aeruginosa and at 4 μM against X. axonopodis. Nine peptide variants (SEQ ID NO 6, SEQ ID NO 7, SEQ ID NO 8, SEQ ID NO 9, SEQ ID NO 10, SEQ ID NO 11, SEQ ID NO 12, SEQ ID NO 13, and SEQ ID NO 14) from these screens demonstrated increased antimicrobial activity against one or more bacteria. All peptides exhibited low to no cytotoxicity after overnight incubation on HaCaT cells. However, peptides 52, 90, 91, 93, and 94 (represented by SEQ ID NO 7, SEQ ID NO 9, SEQ ID NO 10, SEQ ID NO 12, and SEQ ID NO 13, respectively) exhibited low levels of hemolysis at higher concentrations. Peptides 20, 60, 92, and 96 (SEQ ID NO 6, SEQ ID NO 8, SEQ ID NO 11, and SEQ ID NO 14, respectively) showed no significant changes in hemolysis when compared to the PBS control.

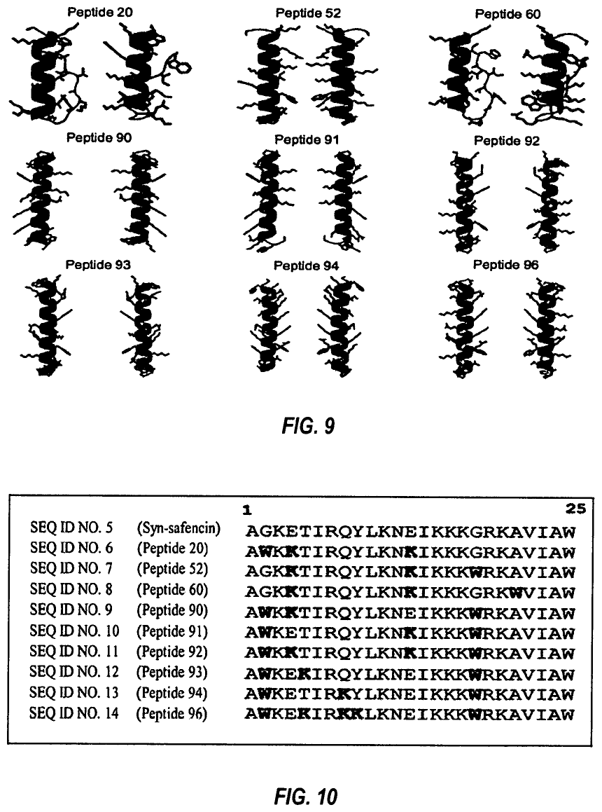

As shown in FIG. 9, secondary structure predictions using PEP FOLD software confirmed that each of the selected peptide variants retained a helical structure; however, all peptides with the G18W replacement existed as a full alpha helix, while peptides without this mutation existed as an N-terminal coil with a C-terminal helix. For ease of illustration, FIG. 10 shows the amino acid sequence alignment of the truncated syn-safencin peptide fragment and the syn-safencin peptide variants 20, 52, 60, 90, 91, 92, 93, 94, and 96 with residue changes between the truncated syn-safencin and each peptide variant being highlighted in red. The secondary structure predictions of all syn-safencin peptide variants revealed a clustering of the hydrophobic residues on one side of the helix with most of the charged residues localizing to the other side of the helix (as shown in FIG. 9 with the hydrophobic, cationic, and anionic residues within each peptide variant being shown in orange, blue, and red, respectively).

To assess if these peptides were more active than the parent peptide fragment (syn-safencin), additional MIC assays were conducted on E. coli, X. axonopodis, P. aeruginosa, and S. pyogenes. The results are summarized in Table 1. MICs as low as 250 nM were observed for the optimized synthetic variant peptides, making these optimized peptides more potent than the original syn-safencin peptide.

TABLE 1
MICs of syn-safencin library peptides

| | | Gram (−) | | | Gram (+) |
|---|---|---|---|---|---|
| | Pep- | E. | X. | P. | S. |
| Sequence # | tide # | coli | axonopodis | aeruginosa | pyogenes |
| SEQ ID NO. 6 | 20 | 8 μM | 1 μM | 10 μM | 8 μM |
| SEQ ID NO. 7 | 52 | 8 μM | 1 μM | 10 μM | 8 μM |
| SEQ ID NO. 8 | 60 | N/A | 2 μM | N/A | N/A |
| SEQ ID NO. 9 | 90 | 2 μM | 1 μM | N/A | 8 μM |
| SEQ ID NO. 10 | 91 | 2 μM | 1 μM | N/A | 10 μM |
| SEQ ID NO. 11 | 92 | 4 μM | 1 μM | N/A | 8 μM |
| SEQ ID NO. 12 | 93 | 1 μM | 2 μM | N/A | 8 μM |
| SEQ ID NO. 13 | 94 | 2 μM | 2 μM | 8 μM | 10 μM |
| SEQ ID NO. 14 | 96 | 2 μM | .25 μM | 8 μM | 8 μM |

Previous studies have hypothesized that the cationic stretch of amino acids in helices four and five of enterocin AS-48 may serve as its putative membrane interacting region. However, peptide fragments consisting of only these residues did not exhibit any antimicrobial activity in previous reports. In a study published by the inventors (The Journal of Antibiotics (2018) 74:592-600, incorporated herein by reference in its entirety), which is summarized in part herein, a new peptide was designed and termed syn-safencin that includes a cluster of cationic residues spanning helices four and five and is homologous to enterocin AS-48. The incorporation of cationic residues and preservation of hydrophobic amino acids can be important to the retention of antimicrobial activity in linear syn-safencin peptide variants. Additionally, hydrophobic and cationic residues within the putative membrane interacting region of helices four and five can be important for the antimicrobial activity of the disclosed linear syn-safencin peptide variants.

While the foregoing is focused on a particular instance of bacteriocin identification and peptide fragment modification, the general structural features of syn-safencin allow for the optimization of antibacterial activity using a systematic amino acid substitution approach, such as that discussed and illustrated in FIG. 1. Synthetic AMP variants can be enriched for cationic and hydrophobic residues and by modifying the charge and hydrophobicity of synthetic peptide variants, antimicrobial activity of the peptide could be increased. In some embodiments, the substitution of glycine for tryptophan can extend a predicted or actual helix and may increase the overall helical propensity of the peptide, which may be correlated to an increased antimicrobial activity of the peptide variant.

In some embodiments, increasing the positive charge and localizing it to one face of the helical peptide can be achieved through substitution of glutamic acid for lysine. Increased antimicrobial activity of such peptides may be due to an increased positive charge on the hydrophilic face of the helix. Secondary structure models of these optimized peptides show an idealized amphipathic nature, which can contribute to increased antimicrobial activity.

### Pharmaceutical Compositions and Exemplary Uses of Synthetic AMPs

Synthetic antimicrobial peptides generated or designed according to one or more of the methods disclosed herein can be used to treat or prevent (e.g., prophylactically) an infection caused by target bacteria. Inclusive—and exemplary—of this are the synthetic peptide variants specifically disclosed herein.

For example, a synthetic peptide generated or designed according to one or more of the methods disclosed herein can be used as a pharmaceutical composition to treat an infection caused by or including strains of E. coli, S. pyogenes, S. aureus, P. syringae, P. aeruginosa, X. axonopodis, or combinations thereof. This can include, more particularly, reducing or eliminating an infection of one or more components of a plant caused by X. axonopodis or reducing or eliminating an infection in humans caused by pathogenic or opportunistic bacterial strains. This can include, for example, skin infections caused by S. aureus (e.g., boils or lesions caused by methicillin-resistant S. aurerus) S. pyogenes, or P. aeruginosa (e.g., in burn victims), gastrointestinal and extraintestinal infections (e.g., urinary tract infections and sepsis) caused by pathogenic strains of E. coli.

While it is possible for the synthetic peptides described herein to be administered alone, it may be preferable to formulate the synthetic peptides as pharmaceutical compositions (e.g., formulations). As such, in yet another aspect, pharmaceutical compositions useful in the uses of synthetic peptides are provided. A pharmaceutical composition is any composition that may be administered in vitro or in vivo or both to a subject to treat or ameliorate a condition. In a preferred embodiment, a pharmaceutical composition may be administered in vivo. A subject may include one or more cells or tissues, or organisms. In some exemplary embodiments, the subject is a plant or animal. In some embodiments, the animal is a mammal. The mammal may be a human or primate in some embodiments. A mammal includes any mammal, such as by way of non-limiting example, cattle, pigs, sheep, goats, horses, camels, buffalo, cats, dogs, rats, mice, and humans.

As used herein the terms “pharmaceutically acceptable” and “physiologically acceptable” mean a biologically compatible formulation, gaseous, liquid or solid, or mixture thereof, which is suitable for one or more routes of administration, in vivo delivery, or contact. A formulation is compatible in that it does not destroy activity of an active ingredient therein (e.g., the synthetic peptides designed and/or optimized according to methods disclosed herein) or induce adverse side effects that outweigh any prophylactic or therapeutic effect or benefit.

In an embodiment, the pharmaceutical compositions may be formulated with pharmaceutically acceptable excipients such as carriers, solvents, stabilizers, adjuvants, diluents, etc., depending upon the particular mode of administration and dosage form. The pharmaceutical compositions should generally be formulated to achieve a physiologically compatible pH and may range from a pH of about 3 to a pH of about 11, preferably about pH 3 to about pH 7, depending on the formulation and route of administration. In alternative embodiments, it may be preferred that the pH is adjusted to a range from about pH 5 to about pH 8. More particularly, the pharmaceutical compositions may comprise a therapeutically or prophylactically effective amount of at least one compound as described herein, together with one or more pharmaceutically acceptable excipients. Optionally, the pharmaceutical compositions may comprise a combination of the compounds described herein or may include a second active ingredient useful in the treatment or prevention of a bacterial infection (e.g., an antibiotic).

Formulations, for example, for parenteral or oral administration, are most typically solids, liquid solutions, emulsions or suspensions, while inhalable formulations for pulmonary administration are generally liquids or powders, with powder formulations being generally preferred. A preferred pharmaceutical composition may also be formulated as a lyophilized solid that is reconstituted with a physiologically compatible solvent prior to administration. Alternative pharmaceutical compositions may be formulated as syrups, creams, ointments, tablets, and the like.

Compositions may contain one or more excipients. Pharmaceutically acceptable excipients are determined in part by the particular composition being administered, as well as by the particular method used to administer the composition. Accordingly, there exists a wide variety of suitable formulations of pharmaceutical compositions (see, e.g., Remington's Pharmaceutical Sciences, incorporated herein by reference).

Suitable excipients may be carrier molecules that include large, slowly metabolized macromolecules such as proteins, polysaccharides, polylactic acids, polyglycolic acids, polymeric amino acids, amino acid copolymers, and inactive virus particles. Other exemplary excipients include antioxidants such as ascorbic acid; chelating agents such as EDTA; carbohydrates such as dextrin, hydroxyalkylcellulose, hydroxyalkylmethylcellulose, stearic acid; liquids such as oils, water, saline, glycerol and ethanol; wetting or emulsifying agents; pH buffering substances; and the like. Liposomes are also included within the definition of pharmaceutically acceptable excipients.

The pharmaceutical compositions described herein may be formulated in any form suitable for the intended method of administration. When intended for oral use, for example, tablets, troches, lozenges, aqueous or oil suspensions, non-aqueous solutions, dispersible powders or granules (including micronized particles or nanoparticles), emulsions, hard or soft capsules, syrups, or elixirs may be prepared. Compositions intended for oral use may be prepared according to any method known to the art for the manufacture of pharmaceutical compositions, and such compositions may contain one or more agents including sweetening agents, flavoring agents, coloring agents, and preserving agents to provide a palatable preparation.

Pharmaceutically acceptable excipients particularly suitable for use in conjunction with tablets include, for example, inert diluents, such as celluloses, calcium or sodium carbonate, lactose, calcium or sodium phosphate; disintegrating agents, such as cross-linked povidone, maize starch, or alginic acid; binding agents, such as povidone, starch, gelatin, or acacia; and lubricating agents, such as magnesium stearate, stearic acid, or talc.

Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example celluloses, lactose, calcium phosphate or kaolin, or as soft gelatin capsules wherein the active ingredient is mixed with non-aqueous or oil medium, such as glycerin, propylene glycol, polyethylene glycol, peanut oil, liquid paraffin or olive oil.

In another embodiment, pharmaceutical compositions may be formulated as suspensions comprising a compound of the embodiments in admixture with at least one pharmaceutically acceptable excipient suitable for the manufacture of a suspension.

In yet another embodiment, pharmaceutical compositions may be formulated as dispersible powders and granules suitable for preparation of a suspension by the addition of suitable excipients.

Excipients suitable for use in connection with suspensions include suspending agents, such as sodium carboxymethylcellulose, methylcellulose, hydroxypropyl methylcellulose, sodium alginate, polyvinylpyrrolidone, gum tragacanth, gum acacia, dispersing, or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycethanol), a condensation product of ethylene oxide with a partial ester derived from a fatty acid and a hexitol anhydride (e.g., polyoxyethylene sorbitan monooleate); polysaccharides and polysaccharide-like compounds (e.g., dextran sulfate); glycoaminoglycans and glycosaminoglycan-like compounds (e.g., hyaluronic acid); and thickening agents, such as carbomer, beeswax, hard paraffin, or cetyl alcohol. The suspensions may also contain one or more preservatives such as acetic acid, methyl and/or n-propyl p-hydroxy-benzoate; one or more coloring agents; one or more flavoring agents; and one or more sweetening agents such as sucrose or saccharin.

The pharmaceutical compositions may also be in the form of oil-in water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth; naturally occurring phosphatides, such as soybean lecithin, esters, or partial esters derived from fatty acids; hexitol anhydrides, such as sorbitan monooleate; and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol, or sucrose. Such formulations may also contain a demulcent, a preservative, a flavoring, or a coloring agent.

Additionally, the pharmaceutical compositions may be in the form of a sterile injectable preparation, such as a sterile injectable aqueous emulsion or oleaginous suspension. This emulsion or suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,2-propane-diol.

The sterile injectable preparation may also be prepared as a lyophilized powder. Among the acceptable vehicles and solvents that may be employed are water, Ringer's solution, and an isotonic sodium chloride solution. In addition, sterile fixed oils may be employed as a solvent or suspending medium. For this purpose, any bland fixed oil may be employed including synthetic mono- or diglycerides. In addition, fatty acids such as oleic acid may be used in the preparation of injectables.

Alternatively, or in addition, non-biochemical compounds can be added to the pharmaceutical compositions to reduce the toxicity of the therapeutic and/or improve the half-life. Suitable amounts and ratios of an additive that can reduce toxicity can be determined via a cellular assay. With respect to the active ingredient (e.g., a synthetic AMP), toxicity reducing compounds can be added to the pharmaceutical composition as 0.25, 0.5, 0.75, 1, 1.5, 2, 2.5, 3, 3.5, 4, 4.5, 5, 10, 20, 50, or 100 weight equivalents, or a range bounded by any two of the aforementioned numbers, or about any of the numbers. In some embodiments, the toxicity reducing compound is a cocoamphodiacetate such as Miranol® (disodium cocoamphodiacetate). In other embodiments, the toxicity reducing compound is an amphoteric surfactant. In some embodiments, the toxicity reducing compound is a surfactant. In other embodiments, the molar ratio of cocoamphodiacetate to active ingredient is between about 8:1 and 1:1, preferably about 4:1. In some embodiments, the toxicity reducing compound is allantoin.

In some embodiments, a pharmaceutical composition is prepared utilizing one or more sufactants. In an exemplary embodiment, the active ingredient (e.g., a synthetic AMP) is complexed with one or more poloxamer surfactants. Poloxamer surfactants are nonionic triblock copolymers composed of a central hydrophobic chain of polyoxypropylene (poly(propylene oxide)) flanked by two hydrophilic chains of polyoxyethylene (poly(ethylene oxide)). In some embodiments, the poloxamer is a liquid, paste, or flake (solid). Examples of suitable poloxamers include those by the trade names Synperonics, Pluronics, or Kolliphor.

In some embodiments, one or more of the poloxamer surfactant in the composition is a flake poloxamer. In some embodiments, the one or more poloxamer surfactant in the composition has a molecular weight of about 3600 g/mol for the central hydrophobic chain of polyoxypropylene and has about 70% polyoxyethylene content. In some embodiments, the ratio of the one or more poloxamer to active ingredient (e.g., a synthetic AMP) is between about 50 to 1; about 40 to 1; about 30 to 1; about 20 to 1; about 10 to 1; about 5 to 1; about 1 to 1; about 1 to 10; about 1 to 20; about 1 to 30; about 1 to 40; or about 1 to 50. In other embodiments, the ratio of the one or more poloxamer to active ingredient (e.g., synthetic AMPs) is between 50 to 1; 40 to 1; 30 to 1; 20 to 1; 10 to 1; 5 to 1; 1 to 1; 1 to 10; 1 to 20; 1 to 30; 1 to 40; or 1 to 50. In some embodiments, the ratio of the one or more poloxamer to active ingredient (e.g., synthetic AMPs) is between about 50 to 1 to about 1 to 50. In other embodiments, the ratio of the one or more poloxamer to active ingredient (e.g., a synthetic AMP) is between about 30 to 1 to about 3 to 1. In some embodiments, the poloxamer is Pluronic F127.

The amount of poloxamer may be based upon a weight percentage of the composition. In some embodiments, the amount of poloxamer is about 10%, 15%, 20%, 25%, 30%, 35%, 40%, about any of the aforementioned numbers, or a range bounded by any two of the aforementioned numbers or the formulation. In some embodiments, the one or more poloxamer is between about 10% to about 40% by weight of a formulation administered to the patient. In some embodiments, the one or more poloxamer is between about 20% to about 30% by weight of the formulation. In some embodiments, the formulation contains less than about 50%, 40%, 30%, 20%, 10%, 5%, or 1% of active ingredient, or about any of the aforementioned numbers. In some embodiments, the formulation contains less than about 20% by weight of active ingredient (e.g., a synthetic AMP).

The above described poloxamer formulations are particularly suited for the methods of treatment, device coatings, preparation of unit dosage forms (e.g., solutions, mouthwashes, injectables), etc.

In one embodiment, the compounds described herein may be formulated for oral administration in a lipid-based formulation suitable for low solubility compounds. Lipid-based formulations can generally enhance the oral bioavailability of such compounds.

As such, in some embodiments, a therapeutically or prophylactically effective amount of a compound described herein can be combined together with at least one pharmaceutically acceptable excipient selected from the group consisting of medium chain fatty acids or propylene glycol esters thereof (e.g., propylene glycol esters of edible fatty acids such as caprylic and capric fatty acids) and pharmaceutically acceptable surfactants such as polyoxyl 40 hydrogenated castor oil.

In an alternative embodiment, cyclodextrins may be added as aqueous solubility enhancers. Preferred cyclodextrins include hydroxypropyl, hydroxyethyl, glucosyl, maltosyl and maltotriosyl derivatives of □-, □-, and □-cyclodextrin. A particularly preferred cyclodextrin solubility enhancer is hydroxypropyl-o-cyclodextrin (BPBC), which may be added to any of the above-described compositions to further improve the aqueous solubility characteristics of the compounds of the embodiments. In one embodiment, the composition comprises about 0.1% to about 20% hydroxypropyl-o-cyclodextrin, more preferably about 1% to about 15% hydroxypropyl-o-cyclodextrin, and even more preferably from about 2.5% to about 10% hydroxypropyl-o-cyclodextrin. The amount of solubility enhancer employed will depend on the amount of the compound of the embodiments in the composition.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

A pharmaceutical composition and/or formulation contains a total amount of the active ingredient(s) sufficient to achieve an intended therapeutic effect.

### Dosages

The pharmaceutical compositions may, for convenience, be prepared or provided as a unit dosage form. Preparation techniques include bringing into association the active ingredient (e.g., synthetic AMPs) and pharmaceutical carrier(s) and/or excipient(s). In general, pharmaceutical compositions are prepared by uniformly and intimately associating the active ingredient with liquid carriers or finely divided solid carriers or both, and then, if necessary, shaping the product. For example, a tablet may be made by compression or molding. Compressed tablets may be prepared by compressing, in a suitable machine, an active ingredient (e.g., synthetic AMPs) in a free-flowing form such as a powder or granules, optionally mixed with a binder, lubricant, inert diluent, preservative, surface-active or dispersing agent. Molded tablets may be produced by molding, in a suitable apparatus, a mixture of powdered compound (e.g., synthetic AMPs) moistened with an inert liquid diluent. The tablets may optionally be coated or scored and may be formulated so as to provide a slow or controlled release of the active ingredient therein.

Compounds (e.g., synthetic AMPs), including pharmaceutical compositions can be packaged in unit dosage forms for ease of administration and uniformity of dosage. A “unit dosage form” as used herein refers to a physically discrete unit suited as unitary dosages for the subject to be treated; each unit containing a predetermined quantity of compound optionally in association with a pharmaceutical carrier (e.g., excipient, diluent, vehicle or filling agent) which, when administered in one or more doses, is calculated to produce a desired effect (e.g., prophylactic or therapeutic effect or benefit). Unit dosage forms can contain a daily dose or unit, daily sub-dose, or an appropriate fraction thereof, of an administered compound. Unit dosage forms also include, for example, capsules, troches, cachets, lozenges, tablets, ampules and vials, which may include a composition in a freeze-dried or lyophilized state; a sterile liquid carrier, for example, can be added prior to administration or delivery in vivo. Unit dosage forms additionally include, for example, ampules and vials with liquid compositions disposed therein. Unit dosage forms further include compounds for transdermal administration, such as “patches” that contact with the epidermis of the subject for an extended or brief period of time. The individual unit dosage forms can be included in multi-dose kits or containers. Pharmaceutical formulations can be packaged in single or multiple unit dosage forms for ease of administration and uniformity of dosage.

Compounds (e.g., synthetic AMPs) can be administered in accordance with the methods at any frequency as a single bolus or multiple dose e.g., one, two, three, four, five, or more times hourly, daily, weekly, monthly, or annually or between about 1 to 10 days, weeks, months, or for as long as appropriate. Exemplary frequencies are typically from 1-7 times, 1-5 times, 1-3 times, 2-times or once, daily, weekly or monthly. For example, twice weekly for two weeks. Timing of contact, administration ex vivo or in vivo can be dictated by the infection, pathogenesis, symptom, pathology, or adverse side effect to be treated. For example, an amount can be administered to the subject substantially contemporaneously with, or within about 1-60 minutes or hours of the onset of a symptom or adverse side effect, pathogenesis, or vaccination. Long-acting pharmaceutical compositions may be administered twice a day, once a day, once every two days, two times a week, three times a week, twice a week, every 3 to 4 days, or every week depending on half-life and clearance rate of the particular formulation. For example, in an embodiment, a pharmaceutical composition contains an amount of a compound as described herein that is selected for administration to a patient on a schedule selected from: twice a day, once a day, once every two days, three times a week, twice a week, and once a week.

Localized delivery is also contemplated, including but not limited to delivery techniques in which the compound is implanted, injected, infused, or otherwise locally delivered. Localized delivery is characterized by higher concentrations of drug at the site of desired action (e.g., the tumor or organ to be treated) versus systemic concentrations of the drug. Well-known localized delivery forms can be used, including long-acting injections; infusion directly into the site of action; depot delivery forms; controlled or sustained delivery compositions; transdermal patches; infusion pumps; and the like. The active ingredient (e.g., synthetic AMPs) can further be incorporated into a biodegradable or bioerodible material or be put into or on a medical device.

Doses may vary depending upon whether the treatment is therapeutic or prophylactic, the onset, progression, severity, frequency, duration, probability of or susceptibility of the symptom, the type pathogenesis to which treatment is directed, clinical endpoint desired, previous, simultaneous or subsequent treatments, general health, age, gender or race of the subject, bioavailability, potential adverse systemic, regional or local side effects, the presence of other disorders or diseases in the subject, and other factors that will be appreciated by the skilled artisan (e.g., medical or familial history). Dose amount, frequency or duration may be increased or reduced, as indicated by the clinical outcome desired, status of the symptom(s) or pathology, and any adverse side effects of the treatment or therapy. The skilled artisan will appreciate the factors that may influence the dosage, frequency, and timing required to provide an amount sufficient or effective for providing a prophylactic or therapeutic effect or benefit. The exact dosage will be determined by the practitioner, in light of factors related to the subject that requires treatment. Dosage and administration are adjusted to provide sufficient levels of the active agent(s) or to maintain the desired effect.

The dosage may range broadly, depending upon the desired effects and the therapeutic indication. Alternatively, dosages may be based and calculated upon the per unit weight of the patient, as understood by those of skill in the art. Although the exact dosage will be determined on a drug-by-drug basis, in most cases, some generalizations regarding the dosage can be made. The systemic daily dosage regimen for an adult human patient may be, for example, an oral dose of between 0.01 mg and 3000 mg of the active ingredient, preferably between 1 mg and 700 mg, e.g. 5 to 200 mg. In some embodiments, the daily dosage regimen is 1 mg, 5 mg, 10, mg, 25 mg, 50 mg, 75 mg, 100 mg, 150 mg, 200 mg, 250 mg, 300 mg, 350 mg, 400 mg, 450 mg, 500 mg, 550 mg, 600 mg, 650 mg, 700 mg, or about any of the aforementioned numbers or a range bounded by any two of the aforementioned numbers. The dosage may be a single one or a series of two or more given in the course of one or more days, as is needed by the subject. In some embodiments, the compounds will be administered for a period of continuous therapy, for example for a week or more, or for months or years. Doses tailored for particular types of bacterial infections or particular patients can be selected based, in part, on the MIC values determined or predicted for the particular type of bacterium/bacteria causative or present in the infection. Particularly preferred formulations for oral dosage include tablet or solutions, particularly solutions compatible with IV administration or solutions compatible with oral administration/use.

In instances where human dosages for compounds have been established for at least some condition, those same dosages may be used, or dosages that are between about 0.1% and 500%, more preferably between about 25% and 250% of the established human dosage. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

In cases of administration of a pharmaceutically acceptable salt, dosages may be calculated as the free base. As will be understood by those of skill in the art, in certain situations it may be necessary to administer the compounds disclosed herein in amounts that exceed, or even far exceed, the above-stated, preferred dosage range in order to effectively and aggressively treat particularly aggressive conditions.

Dosage amount and interval may be adjusted individually to provide plasma levels of the active moiety which are sufficient to maintain the modulating effects, or minimal effective concentration (MEC). For example, therapeutic dosages may result in plasma levels of 0.05 μg/mL, 0.1 μg/mL, 0.5 μg/mL, 1 μg/mL, 5 μg/mL, 10 μg/mL, 15 μg/mL, 20 μg/mL, 25 μg/mL, 30 μg/mL, 35 μg/mL, 40 μg/mL, 45 μg/mL, 50 μg/mL, 55 μg/mL, 60 μg/mL, 65 μg/mL, 70 μg/mL, 75 μg/mL, 80 μg/mL, 85 μg/mL, 90 μg/mL, 95 μg/mL, 100 μg/mL, a range bounded by any two of the aforementioned numbers, or about any of the aforementioned numbers and ranges. In some embodiments, the therapeutic dose is sufficient to establish plasma levels in the range of about 0.1 μg/mL to about 10 μg/mL. In other embodiments, the therapeutic dose is sufficient to establish plasma levels in the range of 1 μg/mL to 20 μg/mL. The MEC may vary for each compound but can be estimated from in vitro or ex vivo data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

Compounds disclosed herein can be evaluated for efficacy and toxicity using known methods. For example, the toxicology of a particular compound, or of a subset of the compounds, sharing certain chemical moieties, may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

As described herein, the methods of the embodiments also include the use of a compound or compounds as described herein together with one or more additional therapeutic agents for the treatment of disease conditions. Thus, for example, the combination of active ingredients may be: (1) co-formulated and administered or delivered simultaneously in a combined formulation; (2) delivered by alternation or in parallel as separate formulations; or (3) by any other combination therapy regimen known in the art. When delivered in alternation therapy, the methods described herein may comprise administering or delivering the active ingredients sequentially (e.g., in separate solution, emulsion, suspension, tablets, pills or capsules) or by different injections in separate syringes. In general, during alternation therapy, an effective dosage of each active ingredient is administered sequentially (e.g., serially), whereas in simultaneous therapy, effective dosages of two or more active ingredients are administered together. Various sequences of intermittent combination therapy may also be used.

As used herein, all numerical values or numerical ranges include integers within such ranges and fractions of the values or the integers within ranges unless the context clearly indicates otherwise. Thus, to illustrate, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. Reference to a range of 0-72 h, includes 1, 2, 3, 4, 5, 6, 7 h, etc., as well as 1, 2, 3, 4, 5, 6, 7 minutes, etc., and so forth. Reference to a range of 0-72 h, includes 1, 2, 3, 4, 5, 6, 7 h, etc., as well as 1, 2, 3, 4, 5, 6, 7 minutes, etc., and so forth. Reference to a range of doses, such as 0.1-1 μg/kg, 1-10 μg/kg, 10-25 μg/kg, 25-50 μg/kg, 50-100 μg/kg, 100-500 μg/kg, 500-1,000 μg/kg, 1-5 mg/kg, 5-10 mg/kg, 10-20 mg/kg, 20-50 mg/kg, 50-100 mg/kg, 100-250 mg/kg, 250-500 mg/kg, includes 0.11-0.9 μg/kg, 2-9 μg/kg, 11.5-24.5 μg/kg, 26-49 μg/kg, 55-90 μg/kg, 125-400 μg/kg, 750-800 μg/kg, 1.1-4.9 mg/kg, 6-9 mg/kg, 11.5-19.5 mg/kg, 21-49 mg/kg, 55-90 mg/kg, 125-200 mg/kg, 275.5-450.1 mg/kg, etc. A series of ranges, for example, 1-10 μg/kg, 10-25 μg/kg, 25-50 μg/kg, 50-100 μg/kg, 100-500 μg/kg, 500-1,000 μg/kg, 1-5 mg/kg, 5-10 mg/kg, 10-20 mg/kg, 20-50 mg/kg, 50-100 mg/kg, 100-250 mg/kg, 250-500 mg/kg, includes 1-25 μg/kg, 10-25 μg/kg, 25-100 μg/kg, 100-1,000 μg/kg, 1-10 mg/kg, 1-20 mg/kg etc.

### Co-Administration

As used herein, “co-administration” means concurrently or administering one substance followed by beginning the administration of a second substance within 24 hours, 20 hours, 16 hours, 12 hours, 8 hours, 4 hours, 1 hour, 30 minutes, 15 minutes, 5 minutes, 1 minute, a range bounded by any two of the aforementioned numbers, and/or about any of the aforementioned numbers. In some embodiments, co-administration is concurrent.

In some embodiments, two or more synthetic antimicrobial peptides are co-administered. In some embodiments, one or more synthetic antimicrobial peptides are co-administered with one or more antibiotics. In some embodiments, the co-administration of p synthetic antimicrobial peptide(s) with or without antibiotic(s) accounts for the therapeutic benefit.

Some embodiments are directed to the use of companion diagnostics to identify an appropriate treatment for the patient. A companion diagnostic is an in vitro diagnostic test or device that provides information that is highly beneficial, or in some instances essential, for the safe and effective use of a corresponding therapeutic composition. Such tests or devices can identify patients likely to be at risk for adverse reactions as a result of treatment with a particular therapeutic composition. Such tests or devices can also monitor responsiveness to treatment (or estimate responsiveness to possible treatments). Such monitoring may include schedule, dose, discontinuation, or combinations of therapeutic compositions. In some embodiments, the synthetic antimicrobial peptide is selected by identifying a bacterial biomarker at the infection site. The term biomarker includes, but is not limited to, genetic elements (e.g., presence/absence of a mutation and/or increase/decrease in expression level of a genetic element), proteins (e.g., presence/absence of a sequence/conformational mutation and/or increase/decrease in expression level of a protein), and cellular responses, such as cytotoxicity.

### Select Definitions of the Present Disclosure

Unless defined otherwise, all technical and scientific terms used herein have the same meaning as commonly understood by one of ordinary skill in the art to which the present disclosure pertains.

The term “sequence identity,” as used herein, refers to two amino acid sequences or subsequences that are identical or that have a specified percentage of amino acid residues that are the same (e.g., 60% or 65% identity, preferably, 70%-95% identity, more preferably, >95% identity), when compared and aligned for maximum correspondence over a window of comparison, or over a designated region, as measured using a sequence comparison algorithm as known in the art or by manual alignment and visual inspection. In certain embodiments, the described identity exists over a region that is at least about 5 to 10 amino acids in length.

A “substitution” at a certain amino acid position or residue can be a change into any of the other 19 naturally-occurring amino acids and can be made via direct peptide synthesis, by changing the nucleotide sequence corresponding to the amino acid codon, or by any other molecular technique known in the art. In some instances, an amino acid substitution can be a “conservative substitution.” As used herein, the term “conservative substitution” refers to the well-established principle of protein chemistry that “conservative amino acid substitutions” can frequently be made in a protein without altering either the conformation or the function of the protein. Such changes include substituting any of isoleucine, valine, and leucine for any other of these hydrophobic amino acids; aspartic acid for glutamic acid and vice versa; glutamine for asparagine and vice versa; and serine for threonine and vice versa. Substituting any of tryptophan, tyrosine, and phenylalanine for any other of these aromatic amino acids and vice versa. Other substitutions can also be considered conservative, depending on the environment of the particular amino acid and its role in the three-dimensional structure of the protein. For example, glycine and alanine can frequently be interchangeable, as can alanine and valine. Methionine, which is relatively hydrophobic, can frequently be interchanged with leucine and isoleucine, and sometimes with valine. Lysine and arginine are frequently interchangeable in locations in which the significant feature of the amino acid residue is its charge, as the differing pKs of these two amino acid residues is not significant.

As used herein, the terms “anionic residue,” “acidic residue,” or similar reference the amino acids glutamic acid and aspartic acid.

The terms “cationic residue,” “basic residue,” or similar reference the amino acids lysine, arginine, and histidine. A “cluster of cationic residues” is defined as a continuous stretch of amino acids having an overall charge—at pH=7—that is greater than or equal to +2. The length of the continuous stretch of amino acids when identifying a “cluster of cationic residues” is understood to be at most about 30 amino acids, preferably at most about 25 amino acids or between about 20-25 amino acids.

The terms “hydrophobic residue,” “nonpolar residue,” or similar preferably includes amino acids valine, leucine, isoleucine, phenylalanine, and tryptophan, but more generally includes glycine, alanine, and methionine. It should be appreciated that the presence of the former hydrophobic residues in an antimicrobial peptide can, in some embodiments, correlate with increased antimicrobial activity compared to the presence of the latter hydrophobic residues. Accordingly, when determining the hydrophobicity of a peptide, as described herein, any of the foregoing hydrophobic residues are included in the total number of hydrophobic residues, though it should be appreciated that a higher proportion of preferred hydrophobic residues can make a qualitative difference in the hydrophobicity of the peptide and/or a quantitative difference in the antimicrobial activity associated therewith.

The term “polar residue” or similar includes amino acids serine, threonine, cysteine, tyrosine, asparagine, and glutamine.

As used herein, a “short-chained amino acid,” “short-chained aliphatic residue” or similar includes glycine, alanine, and valine, preferably glycine and alanine, more preferably glycine.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present embodiments. At the very least, and not as an attempt to limit the application of the doctrine of equivalents to the scope of the claims, each numerical parameter should be construed in light of the number of significant digits and ordinary rounding approaches.

The term “about,” when used herein to refer to the percentage of hydrophobic residues in a polypeptide should be understood to include those values within 10% of the stated value, preferably within 5% of the stated value, and more preferably within 2% of the stated value.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

While the detailed description is separated into sections, the section headers and contents within each section are not intended to be self-contained descriptions and embodiments. Rather, the contents of each section within the detailed description are intended to be read and understood as a collective whole where elements of one section may pertain to and/or inform other sections. Accordingly, embodiments specifically disclosed within one section may also relate to and/or serve as additional and/or alternative embodiments in another section having the same and/or similar systems, modules, devices, methods, and/or terminology.

Various alterations and/or modifications of the inventive features illustrated herein, and additional applications of the principles illustrated herein, which would occur to one skilled in the relevant art and having possession of this disclosure, can be made to the illustrated embodiments without departing from the spirit and scope of the invention as defined by the claims, and are to be considered within the scope of this disclosure. Thus, while various aspects and embodiments have been disclosed herein, other aspects and embodiments are contemplated. While a number of methods and components similar or equivalent to those described herein can be used to practice embodiments of the present disclosure, only certain components and methods are described herein.

It will also be appreciated that systems, devices, products, kits, methods, and/or processes, according to certain embodiments of the present disclosure may include, incorporate, or otherwise comprise properties, features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

The present disclosure may be embodied in other specific forms without departing from its spirit or essential characteristics. The described embodiments are to be considered in all respects only as illustrative and not restrictive. The scope of the invention is, therefore, indicated by the appended claims rather than by the foregoing description. While certain embodiments and details have been included herein and in the attached disclosure for purposes of illustrating embodiments of the present disclosure, it will be apparent to those skilled in the art that various changes in the methods, products, devices, and apparatus disclosed herein may be made without departing from the scope of the disclosure or of the invention, which is defined in the appended claims. All changes which come within the meaning and range of equivalency of the claims are to be embraced within their scope.

### EXAMPLES

### Example 1

Bacterial strains and growth conditions used in experimental work presented in Examples 2-8:

Bacteria.

E. coli BL-21 frozen stock culture was purchased from Thermo Fischer. Pseudomonas aeruginosa PAO1 frozen stock culture was obtained from the Shrout Laboratory at the University of Notre Dame. Pseudomonas syringae frozen stock culture was obtained from the Innes Laboratory at Indiana University. Xanthomonas axonopodis pathovar Starr and Garces pathovar phaseoli (ATCC 9563) frozen stock culture was purchased from the ATCC. Streptococcus pyogenes M1T1 frozen stock culture was obtained from the lab of Dr. Victor Nizet, University of California San Diego. A methicillin resistant Staphylococcus aureus JKD frozen stock culture was obtained from the lab of Dr. Timothy Stinear, University of Melbourne.

Growth conditions.

The E. coli, P. aeruginosa, and P. syringae bacterial strains were routinely grown in LB broth (EMD Chemicals, Gibbstown, N.J.). S. aureus and S. pyogenes were routinely grown in Todd Hewitt broth (Neogen Corporation, Lansing, Mich.). X. axonopodis was routinely grown in Nutrient Broth (Sigma-Aldrich Co., St. Louis, Mo.). P. syringae and X. axonopodis were grown at room temperature without agitation. All other bacteria were grown at 37° C. with agitation.

### Example 2

MIC determination and peptide screening.

Overnight bacterial cultures were diluted to an OD of 0.01. μL of diluted culture and 10 μL of 10× peptide or vehicle were added to the wells of a 96-well microtiter plate for a final 1× concentration. Bacteria were grown in desired growth conditions in a Synergy H1 Microplate Reader (Biotek, Winooski, Vt.). For MIC, serial two-fold dilutions of the peptides were used. MIC was determined as the concentration of peptide that prevented overnight growth as indicated by OD 600. For peptide screening, 8 μM or 4 μM peptide was screened against various bacteria. Peptides that inhibited overnight growth were selected for further study.

### Example 3

Peptide cytotoxicity assays.

Eukaryotic cytotoxicity was determined by ethidium homodimer and hemolysis assays. Ethidium homodimer assays were carried out with HaCaT cells in 24-well culture dishes grown to 90% confluency. Medium was aspirated, and cells were washed with PBS. Peptide in fresh DMEM was added to the cells at the desired concentration. Cells were incubated with peptide for 16 h. Medium was aspirated, and cells were washed with PBS. Cells were incubated in 4 μM ethidium homodimer (Molecular Probes) in PBS for 30 min. Fluorescence was determined by 528 excitation and 617 nm emission and a cutoff value of 590 nm. Saponin (0.1%) was then added to each well and incubated for 20 min. The fluorescence was read again. Percent membrane permeabilization was determined by dividing the initial fluorescence by the second fluorescence reading. For hemolysis assays, 100 μL of sheep red blood cells (RBCs) were washed three times in cold PBS (Thermo Fischer). Washed cells were resuspended in 25 mL of PBS. Triton, PBS, or peptide in 10% DMSO/PBS were added to 180 μL of resuspended RBCs and incubated at 37° C. for 1 h. Samples were read at 450 nm. Data was expressed as percent hemolysis by relativizing to the Triton and PBS controls.

### Example 4

Circular dichroism spectroscopy.

Peptides were dissolved to a final concentration of 25 μM in the following solvents: 9 mM SDS (Sigma-Aldrich), 50% trifluoroethanol (TFE) (Sigma-Aldrich), and water. For peptide experiments in lipopolysaccharide (LPS) (Sigma-Aldrich), peptide was dissolved to a final concentration of 5 μM in 5 μM LPS. Using a Jasco Circular Dichroism Spectrometer, samples were scanned from 190 to 250 nm at 20 nm/min in a 2 mm cuvette. Data were averaged over three scans and blanks of the solvent were subtracted from the peptide scans.

### Example 5

Fluorescence microscopy.

Overnight culture of BL-21 E. coli were diluted to a starting OD=1. 1 mL of cells were washed three times in saline solution. Cells were then resuspended in 1 mL of saline, 70% isopropyl alcohol, or peptide in saline and incubated at 37° C. for 30 min. Cells were pelleted and resuspended in 1 mL of saline with 1.5 μL/mL propidium iodide (PI) (Thermo Fischer) and incubated at room temperature in the dark for 15 min. Samples were pelleted and washed three times with saline. Samples were resuspended in 1 mL of saline. 5 μL of the sample were imaged on a Nikon microscope using the RFP and DIC channels with a 100 and 300 ms exposure times, respectively.

Flow cytometry.

Cells were prepared using the same protocol for fluorescence microscopy followed by suspension in 200 μL of saline. Samples were run on the Aria flow cytometer (BD Biosciences) and fluorescence intensity was measured using the Texas Red channel.

1. A method for generating synthetic antimicrobial peptides, comprising:
identifying a peptide fragment of an antimicrobial peptide, the peptide fragment comprising a cluster of cationic residues and at least about 25% hydrophobic residues, preferably between about 40%-60% hydrophobic residues; and
generating a peptide variant library based on the peptide fragment by varying a hydrophobicity and charge of residues comprising the peptide fragment.

2. The method of claim 1, wherein identifying the peptide fragment comprises querying a data structure comprising known or putative protein coding sequences, or translated amino acid sequences derived therefrom, using a query input comprising at least a portion of a known or putative bacteriocin to identify one or more homologues thereof.

3. The method of claim 1, wherein identifying the peptide fragment comprises identifying a bacteriocin comprising the peptide fragment, the bacteriocin having an active form wherein a sequence of the peptide fragment in the active form comprises only naturally occurring amino acids.

4. The method of claim 1, wherein identifying the peptide fragment comprises identifying an active form of a bacteriocin comprising the peptide fragment, each residue of the peptide fragment in the active form lacking a posttranslational modification.

5. The method of claim 1, wherein identifying the peptide fragment comprises identifying at least a portion of a class II bacteriocin or a class I circularized bacteriocin.

6. The method of claim 1, wherein varying the hydrophobicity and charge of residues comprises iteratively substituting a lysine residue for each acidic residue and each polar residue within the peptide fragment to generate a primary set of peptide variants.

7. The method of claim 6, further comprising iteratively substituting a tryptophan residue for each short-chained aliphatic residue and each nonpolar residue within the peptide fragment to generate a secondary set of peptide variants and within each peptide variant of the primary set of peptide variants to generate a tertiary set of peptide variants.

8. The method of claim 1, wherein varying the hydrophobicity and charge of residues comprises iteratively substituting a lysine residue for one or more aspartic acid, glutamic acid, glutamine, threonine, and serine residues within the peptide fragment to generate a primary set of peptide variants.

9. The method of claim 8, further comprising iteratively substituting a tryptophan residue at one or more glycine and alanine residues within the peptide fragment to generate a secondary set of peptide variants and within one or more peptide variants of the primary set of peptide variants to generate a tertiary set of peptide variants.

10. The method of claim 1, further comprising assaying one or more peptide variants for increased antimicrobial activity against a target bacterium and/or an increased spectrum of antimicrobial activity.

11. The method of claim 10, wherein at least one peptide variant comprises an increased antimicrobial activity against the target bacterium as compared with a baseline antimicrobial activity of the peptide fragment.

12. The method of claim 1, wherein varying the hydrophobicity and charge of residues comprises increasing a positive charge of a peptide variant of the primary set of peptide variants and localizing the positive charge to a same face of a predicted alpha helix corresponding to a predicted secondary structure for the peptide variant.

13. The method of claim 12, wherein varying the hydrophobicity and charge of residues causes the peptide variant to become amphipathic.

14. A synthetic antimicrobial peptide defined by a sequence selected from the group consisting of: SEQ ID NO 6, SEQ ID NO 7, SEQ ID NO 8, SEQ ID NO 9, SEQ ID NO 10, SEQ ID NO 11, SEQ ID NO 12, SEQ ID NO 13, and SEQ ID NO 14.

15. The synthetic antimicrobial peptide of claim 14 for use in reducing or eliminating an infection of one or more components of a plant caused by thamonas axonopodis .


SEQUENCE LISTING (14 sequences)

SEQ ID NO: 1 - protein, 105 aa, Enterococcus sp.
MVKENKFSKIFILMALSFLGLALFSASLQFLPIAHMAKEFGIPAAVAGTVLNVVEAGGWVTTIVSILTAVGSGGLSLLAAAGRESIKAYLKKEIKKKGKRAVIAW

SEQ ID NO: 2 - protein, 108 aa, Bacillus safensis
MTETRNEIKLHVLFGALAVGFLMLALFSFSLQMLPVADLAKEFGIPGSVAAVVLNVVEAGGAVTTIVSILTAVGSGGLSLIAAAGKETIRQYLKNEIKKKGRKAVIAW

SEQ ID NO: 3 - protein, 38 aa, Bacillus safensis
MTETRNEIKLHVLFGALAVGFLMLALFSFSLQMLPVAD

SEQ ID NO: 4 - protein, 31 aa, Bacillus safensis
LSLIAAAGKETIRQYLKNEIKKKGRKAVIAW

SEQ ID NO: 5 - protein, 25 aa, Bacillus safensis
AGKETIRQYLKNEIKKKGRKAVIAW

SEQ ID NO: 6 - protein, 25 aa, Artificial Sequence
  Peptide 20
AWKKTIRQYLKNKIKKKGRKAVIAW

SEQ ID NO: 7 - protein, 25 aa, Artificial Sequence
  Peptide 52
AGKKTIRQYLKNKIKKKWRKAVIAW

SEQ ID NO: 8 - protein, 25 aa, Artificial Sequence
  Peptide 60
AGKKTIRQYLKNKIKKKGRKWVIAW

SEQ ID NO: 9 - protein, 25 aa, Artificial Sequence
  Peptide 90
AWKKTIRQYLKNEIKKKWRKAVIAW

SEQ ID NO: 10 - protein, 25 aa, Artificial Sequence
  Peptide 91
AWKETIRQYLKNKIKKKWRKAVIAW

SEQ ID NO: 11 - protein, 25 aa, Artificial Sequence
  Peptide 92
AWKKTIRQYLKNKIKKKWRKAVIAW

SEQ ID NO: 12 - protein, 25 aa, Artificial Sequence
  Peptide 93
AWKEKIRQYLKNEIKKKWRKAVIAW

SEQ ID NO: 13 - protein, 25 aa, Artificial Sequence
  Peptide 94
AWKETIRKYLKNEIKKKWRKAVIAW

SEQ ID NO: 14 - protein, 25 aa, Artificial Sequence
  Peptide 96
AWKEKIRKKLKNEIKKKWRKAVIAW